(19) United States
(12) Patent Application Publication
(10) Pub. No.: US 2026/0062379 A1
(43) Pub. Date: Mar. 5, 2026

(54) METHOD FOR TREATING RESIDUE FROM THE PRODUCTION OF ORGANIC ISOCYANATES

(71) Applicant: Covestro Deutschland AG, Leverkusen (DE)

(72) Inventors: Michael Merkel, Düsseldorf (DE); Christoph Eggert, Köln (DE); Ralf Schneider, Leverkusen (DE); Robert Maleika, Düsseldorf (DE); Sebastian Stolz, Köln (DE)

(21) Appl. No.: 19/106,676

(22) PCT Filed: Sep. 7, 2023
(86) PCT No.: PCT/EP2023/074537

§ 371 Date: Feb. 26, 2025

(30) Foreign Application Priority Data
Sep. 9, 2022 (EP) 22194934.0

(51) Int. Cl. C07C 263/20

(52) U.S. Cl. CPC C07C 263/20; C07C 2601/14

(57) ABSTRACT

The invention relates to a method for treating residue from the production of organic isocyanates via the phosgenation of organic amines, comprising a step of mixing a first residue A with a second residue B, obtaining a residue mixture, wherein the residue A is obtained from the production of 1,5-pentane diisocyanate and the residue B comes from the production of an isocyanate that is different from 1,5-pentane diisocyanate. The invention also relates to the use of a first residue A from the production of 1,5-pentane diisocyanate to stabilise a second residue B from the production of at least one organic isocyanate that is different from 1,5-pentane diisocyanate, and stabilised residue mixtures.

The present invention relates to a process for treating residues from the production of organic isocyanates by phosgenation of organic amines. It also relates to the use of a residue from the production of 1,5-pentanediisocyanate and stabilized residue mixtures.

The industrial-scale production of isocyanates by reacting amines with phosgene is known and described in detail in the literature (e.g. Ullmanns Encyklopädie der technischen Chemie, 4th edition, volume 13, pages 347-357, Verlag Chemie, GmbH, D-6940 Weinheim, 1977 or else EP 1 575 908 A1). The production of pure distilled diisocyanates in the distillation processes normally generates a byproduct stream that requires disposal as a residue after the greatest possible distillative removal of free isocyanates.

Such residues always retain reactive groups and there is therefore a risk of exothermic reactions up to and including thermal runaway, i.e. uncontrolled overheating of the exothermic chemical reaction and the technical apparatus in which the residue is stored. In order to avoid this, studies into the thermal stability of the residues are performed with the aim of determining the liberated heat of reaction and the so-called onset temperature, i.e. the temperature above which the reaction commences and liberates energy. The residue may then only be stored for a certain time and at temperatures that are a safety margin below the onset temperature.

Since it is advantageous to handle residues in liquid form and the solidification points increase with increasing concentration of the high-boiling byproducts in the residue, the residues must be diluted to a greater extent in the case of lower onset temperatures and correspondingly lower permissible residue temperatures. Accordingly, either more of the value product remains in the residue or additional measures must be taken, such as for instance the addition of solvents.

In order to avoid unexpected reactions in the residue it is typically desirable to handle different residues separately and not to mix them.

It was accordingly an object of the present invention to provide a process which does not suffer from the aforementioned disadvantages of the prior art and allows improved handling of residue mixtures from the production of organic isocyanates.

The object was achieved according to the invention by a process for the treatment of residues from the production of organic isocyanates by phosgenation of organic amines, comprising a mixing step of a first residue A with a second residue B to obtain a residue mixture, wherein residue A was obtained from the production of 1,5-pentane diisocyanate and residue B was obtained from the production of an isocyanate distinct from 1,5-pentane diisocyanate.

It has now been found that, surprisingly, admixing a residue, preferably a distillation residue, from the production of 1,5-pentane diisocyanate (hereinafter also referred to as pentamethylene diisocyanate or as PDI) with other residues from the production of isocyanates distinct from pentamethylene diisocyanate does not increase the risk of an uncontrolled exothermic reaction but rather often reduces it. This accordingly allows the mixing of such residues without reducing process safety, thus achieving positive technical effects such as for example longer storage stability of the mixed residue or reduced apparatus costs by saving on additional residue reservoirs.

According to the invention the terms “comprising” or “containing” are preferably to be understood as meaning “substantially consisting of” and particularly preferably “consisting of”. The further embodiments recited in the claims and in the description may be combined as desired, provided that the context does not clearly indicate the opposite.

An “organic compound” or “organic radical” contains at least one unit comprising a covalent carbon-hydrogen bond.

The term “aliphatic” is presently defined as meaning non-aromatic hydrocarbon groups that are saturated or unsaturated.

The term “araliphatic” is presently defined as meaning hydrocarbon radicals consisting of both an aromatic hydrocarbon group and a saturated or unsaturated hydrocarbon group which is bonded directly to the aromatic radical.

The term “alicyclic” or “cycloaliphatic” is presently defined as meaning optionally substituted carbocyclic or heterocyclic compounds or units which are not aromatic. “At least one”, as used herein, refers to 1 or more, for example 2, 3, 4, 5, 6, 7, 8, 9 or more. In connection with constituents of the compounds described herein, this figure refers not to the absolute number of molecules, but rather to the nature of the constituent. “At least one isocyanate distinct from pentamethylene diisocyanate” is therefore to be understood as meaning for example that only one type of compound or two or more different types of compounds of this type may be present without specifying the amount of the individual compounds.

Numerical ranges given in the format “in/from x to y” include the values stated. If two or more preferred numerical ranges are given in this format, it is understood that all ranges arising from the combination of the various limits are likewise encompassed.

The process according to the invention is advantageously employable for example when campaigns which each produce different organic isocyanates are run in a production plant for producing organic isocyanates, wherein one of the organic isocyanates is pentamethylene diisocyanate. In the production or the purification of the different organic isocyanates residue A and second residue B are then generated at different times relative to one another and may be mixed to achieve the desired effects, in particular a stabilization, in a residue reservoir for example. There is therefore no need to provide different residue storage means for each different type of residue, or if only one residue reservoir is available this need not be completely emptied, much less washed, when changing campaigns. On the contrary it is possible to always retain a certain amount of residue mixture to allow optimal concentration of the respective residues in the distillation and to benefit from the stabilization due to mixing with 1,5-pentane diisocyanate-based residue.

Another opportunity arises when two production and/or distillation apparatuses for the different organic isocyanates are present at one location. In this case residue A and residue B are generated simultaneously and may be subjected to the mixing step according to the process of the invention. This step may be carried out either in a pipe conduit by means of which the residues are transferred into a collection reservoir, a transport container or into an incineration plant or it is carried out in a residue reservoir. For mixing in the pipe conduit the residues may simply be combined in a pipe conduit. It is preferable when the mixing of residues A and B is improved using a static mixer or a dynamic mixing unit such as for example one or more mixing pumps, dispersers or stirred containers. If the mixing is carried out in a collection reservoir this preferably has respective dedicated inlet ports, optionally with an immersion tube, for the different residues. The mixing is then carried out in the collection reservoir using a stirring means and/or a pumped circulation means which may also be used for temperature control of the residue. It is preferable when the mixing is carried out using a stirring means in the collection reservoir.

Residue A is a liquid residue or a suspension, preferably a liquid residue, obtained in the production of pentamethylene diisocyanate and containing not only PDI but also for example compounds which have a higher boiling point than PDI and were formed on the basis of PDI. These may include for example PDI oligomers such as carbodiimides or trimers, ureas, urethanes, but also chlorinated compounds such as carbamoyl chloride. It is preferable when residue A is a preferably liquid distillation residue obtained at the bottom of a column in which PDI was separated from a PDI-containing substance mixture. In the present case such a residue A is also referred to as “from the production of pentamethylene diisocyanate”.

The residue B is a liquid residue or a suspension, preferably a liquid residue, which was obtained in the production of at least one isocyanate distinct from pentamethylene diisocyanate and which contains not only the at least one isocyanate but also for example compounds which have been formed on the basis of the at least one isocyanate and have a boiling point higher than the respective isocyanate. It is preferably a residue from the production of precisely one isocyanate distinct from PDI. It is preferable when the at least one isocyanate distinct from PDI, in whose production and/or purification process residue B is generated, is at least one aliphatic, cycloaliphatic and/or araliphatic diisocyanate, particularly preferably at least one cycloaliphatic and/or araliphatic diisocyanate. It is more preferable when the at least one isocyanate distinct from PDI is hexamethylene diisocyanate, diisocyanatohexylmethane, isophorone diisocyanate (IPDI), bis(isocyanatomethyl)norbornane (NBDI), 1,3- and 1,4-bis(isocyanatomethyl)cyclohexane and/or xylylene diisocyanate, particularly preferably hexamethylene diisocyanate, diisocyanatohexylmethane, isophorone diisocyanate (IPDI) and/or xylylene diisocyanate, very particularly preferably IPDI and/or xylylene diisocyanate and most preferably IPDI and/or m-xylylene diisocyanate (hereinafter also referred to as XDI). In the present case such a residue B is also referred to as “from the production of at least one isocyanate distinct from pentamethylene diisocyanate”. It is preferable when the at least one isocyanate distinct from PDI is precisely one isocyanate distinct from PDI and optionally isomers thereof and thus also a residue which is based only on this one isocyanate distinct from PDI and optionally isomers thereof.

The production process, i.e. the synthesis per se, and the purification process are both steps which are presently considered as included in the production of an isocyanate, so that in the present case the term “production of an isocyanate” relates not only to the production per se but rather a residue from the production of an isocyanate may likewise also have been generated in the distillation.

In a further embodiment of the process according to the invention residue A and/or residue B, preferably residue A and residue B, are distillation residues.

In a preferred embodiment residue B derives from the production of precisely one aliphatic, cycloaliphatic or araliphatic diisocyanate, preferably precisely one araliphatic or cycloaliphatic diisocyanate, distinct from 1,5-pentane diisocyanate. It is particularly preferable when residue B derives from the production of hexamethylenediisocyanate, diisocyanatohexylmethane, IPDI, bis(isocyanatomethyl)norbornane (NBDI), 1,3- and 1,4-bis(isocyanatomethyl)cyclohexane or xylylene diisocyanate, very particularly preferably from the production of IPDI or xylylene diisocyanate and most preferably from the production of IPDI or XDI.

In order to maximize the yield of value product and thus the economy of the production process it is desirable for residue A and/or residue B to contain very little of the corresponding value product. However, an excessively low residual content of monomeric isocyanate results in a severe viscosity increase up to and including solidification of the residue and so in practice a compromise must always be found. The residues typically contain about 5% to 85% by weight, preferably 10% to 60% by weight, particularly preferably 15% to 50% by weight and very particularly preferably 20% to 40% by weight of monomeric isocyanate upon withdrawal from the production of the isocyanate. The temperature may in principle be selected as desired. A higher temperature of the residue helps to keep the viscosity low and thus maintain pumpability. However, an obstacle to temperature elevation is that the ingredients of the isocyanate residues can undergo exothermic reactions with one another or with themselves. A temperature in the range from 15° C. to 180° C., preferably in the range from 40° C. to 150° C. and particularly preferably in the range from 60° C. to 110° C. has proven to be an advantageous compromise.

It is accordingly also preferable when the temperature in the mixing step is at least periodically, preferably continuously, in the range from 15° C. to 180° C., preferably in the range from 40° C. to 150° C. and particularly preferably in the range from 60° C. to 110° C.

In order to reduce temperature and concentration variations it is preferable to store the at least one residue mixture obtained via the mixing step in at least one residue container, wherein the mixing step may be carried out in a separate mixing apparatus upstream of the residue container or preferably in the residue container itself. The amount of residue mixture present in the residue container thus buffers variations in the newly inflowing residue stream.

The above considerations regarding the preferred temperatures in the mixing step are also applicable to a storage of the at least one residue mixture obtained in the mixing step. The temperature during storage is accordingly at least periodically, preferably continuously, in the range from 15° C. to 180° C., preferably in the range from 40° C. to 150° C. and particularly preferably in the range from 60° C. to 110° C. Particularly during extended storage of the obtained residue mixture it is important to observe the temperature limits since the probability of adverse effects such as solidification of the residue or exothermic decomposition increases with increasing storage duration. If a storage of the residue is carried out it is accordingly advantageous to limit the residence time of the residue in the storage container. The residence time is preferably at most 28 days, particularly preferably at most 7 days and very particularly preferably at most 2 days, most preferably at most 1 day. What is decisive is the calculated residence time which results when the available volume is divided by the volume flow of the residue stream flowing through the residue container. If the inflow and/or outflow of residue occurs discontinuously or with a non-constant volume flow, time-averaged values for the volume flow may be employed.

Storage in a residue container can lead to deposits of residue constituents on the walls of the residue container. There is a high risk of deposits especially in the region of heating or cooling apparatuses.

Sediments which settle on the bottom of the residue container and can lead to clogging may also form. In order to prevent these problems it is advantageous to agitate, preferably to stir, the at least one residue mixture in at least one residue container at least periodically, preferably continuously.

In a further preferred embodiment of the process according to the invention in the mixing step residue B is blended with an efficacious amount of residue A which results in stabilization of residue B, wherein the stabilization preferably entails an increase in the onset temperature relative to residue B alone. Depending on the type of residue B the efficacious amount of residue A may vary. Said amount may be determined for example in preliminary tests by producing mixtures of residue A with the residue B in question in different mixing ratios and then determining their thermal stability by differential scanning calorimetry (DSC) according to DIN EN ISO 11357-1:2017-02. The efficacious amount of residue A may then be directly derived from the mixing ratios for which an increase in the onset temperature for the exothermic reaction of the residue is observed. If two or more exothermic reactions with different onset temperatures occur for a residue B, the one that is decisive is the one having the lowest onset temperature whose release of heat exceeds a value of 100 J/g.

In a further embodiment of the process according to the invention 10 to 80 parts by weight, preferably 30 to 75 parts by weight and particularly preferably 45 to 70 parts by weight of residue A are mixed with 20 to 90 parts by weight, preferably 25 to 70 parts by weight, particularly preferably 30 to 55 parts by weight, of residue B, wherein residue B is in each case a residue from the production of isophorone diisocyanate, bis(isocyanatomethyl)norbornane (NBDI) and/or 1,3- and 1,4-bis(isocyanatomethyl)cyclohexane, preferably from the production of isophorone diisocyanate, or 5 to 30 parts by weight, preferably 7 to 20 parts by weight, of residue A are mixed with 70 to 95 parts by weight, preferably 80 to 93 parts by weight, of residue B, wherein residue B is in each case a residue from the production of xylylene diisocyanate, preferably of m-xylylene diisocyanate.

The residue mixture obtained in the mixing step can be used as an additive to other isocyanates for example. However, due to the variable chemical composition and the variations in properties resulting therefrom, this type of use is limited. In a preferred embodiment of the process according to the invention a thermal recovery of the residue obtained in the mixing step is therefore carried out. To this end said residue may be continuously or discontinuously supplied to an incineration. The advantageous properties achieved of the residue may be utilized via the mixing. The residue may be handled at relatively high temperature and thus relatively low viscosity for example, thus reducing problems due to blockages in the feed lances in the combustion plant for example.

In a further preferred embodiment of the invention monomeric diisocyanates are separated from the residue mixture obtained in the mixing step in a downstream process step. The separation of the monomeric diisocyanates is preferably carried out by producing a solid, preferably free-flowing residue in at least one kneader, shovel, or roller dryer, preferably in a kneader or shovel dryer and particularly preferably in a shovel dryer. The residual mixture obtained in the mixing step is introduced into the dryer optionally together with additives and separated into a gaseous portion containing substantially monomeric diisocyanates, referred to hereinafter as gaseous mixture, and a residue depleted in monomeric diisocyanates. This separation is carried out at negative pressure and elevated temperature, so that here too the advantageous properties of the residue achieved by the mixing step of the process according to the invention may be utilized by allowing the use of a higher temperature for example.

The gaseous mixture of substantially monomeric diisocyanates obtained in this process step may be condensed to obtain a liquid mixture containing substantially pentamethylene diisocyanate and at least one further diisocyanate distinct from pentamethylene diisocyanate. This liquid mixture may be used for producing polyurethanes, polythiourethanes and/or polyureas and is likewise a subject of the present invention. If required the liquid mixture may be subjected to further purification steps, for example distillation, filtration, crystallization and/or extraction, and/or admixed with additives before production of the polyurethanes, polythiourethanes and/or polyureas. It is preferable when the at least one further diisocyanate distinct from pentamethylene diisocyanate in the liquid mixture is hexamethylene diisocyanate, diisocyanatohexylmethane, isophorone diisocyanate (IPDI), bis(isocyanatomethyl)norbornane (NBDI), 1,3- and 1,4-bis(isocyanatomethyl)cyclohexane and/or xylylene diisocyanate, particularly preferably isophorone diisocyanate and/or xylylene diisocyanate and very particularly preferably isophorone diisocyanate and/or m-xylylene diisocyanate. Since the gaseous mixture recovered via the process according to the invention and the inventive liquid mixture of residues derive from the production of organic isocyanates by phosgenation of organic amines, they contain small amounts of additional compounds that are not present in the directly produced process products that are already known. In the context of the liquid mixture according to the invention the term “substantially” is to be understood as meaning that preferably 95% by weight or more, particularly preferably 97% by weight or more and very particularly preferably 99% by weight or more based on the total amount of the mixture are pentamethylene diisocyanate and at least one further diisocyanate distinct from pentamethylene diisocyanate.

The present invention further provides a gaseous or liquid mixture obtainable or produced by the process according to the invention. The gaseous mixture or optionally the liquid mixture may be subjected to distillative workup in a further process step if required to isolate monomeric pentamethylene diisocyanate and the at least one diisocyanate distinct from pentamethylene diisocyanate.

The present invention further provides for the use of a first residue A from the production of 1,5-pentane diisocyanate for stabilizing a second residue B from the production of at least one organic isocyanate distinct from 1,5-pentane diisocyanate, wherein the stabilization preferably entails an increase in the onset temperature relative to residue B alone. It is preferable when the residues in the use according to the invention substantially derive from the production of organic isocyanates by phosgenation of organic amines. Particular preference is given to the use of a first residue A from the production of 1,5-pentane diisocyanate for stabilizing a second residue B from the production of at least one organic isocyanate distinct from 1,5-pentane diisocyanate, wherein the stabilization preferably entails an increase in the onset temperature relative to residue B alone and wherein the residues derive from the production of organic isocyanates by phosgenation of organic amines.

The onset temperatures of the individual residues or different residue mixtures may be determined for example in preliminary tests using differential scanning calorimetry (DSC) according to DIN EN ISO 11357-1:2017-02. The respective onset temperature of the exothermic reaction of the isocyanate residue is decisive. If two or more exothermic reactions with different onset temperatures are observed for a residue, the one that is decisive is the one having the lowest onset temperature whose release of heat exceeds a value of 100 J/g.

In a further preferred embodiment of the use according to the invention, the second residue B is derived from the production of at least one aliphatic, cycloaliphatic or araliphatic diisocyanate, preferably from the production of an araliphatic or cycloaliphatic diisocyanate, distinct from 1,5-pentanediisocyanate, particularly preferably from the production of hexamethylene diisocyanate, diisocyanatohexylmethane, isophorone diisocyanate, bis(isocyanatomethyl)norbornane (NBDI), 1,3- and 1,4-bis(isocyanatomethyl)cyclohexane and/or xylylene diisocyanate, very particularly preferably from the production of IPDI and/or xylylene diisocyanate and most preferably from the production of IPDI and/or XDI.

In a further preferred embodiment of the use according to the invention, residue A and/or residue B, preferably residue A and residue B, are distillation residues. The use according to the invention is particularly suitable for distillation residues because corresponding optimizations may then be performed in the preceding distillation steps. Thus for example the residual monomer content in the bottoms product of the distillation column may be reduced, thus resulting in a reduced loss of value substance or saving a portion of the cooling power required to cool the residues to their respectively allowable storage temperatures.

The present invention further provides a stabilized residue mixture containing 10% to 80% by weight of a first residue A from the production of 1,5-pentane diisocyanate and 20% to 90% by weight of a residue B from the production of isophorone diisocyanate, bis(isocyanatomethyl)norbornane (NBDI) or 1,3- and 1,4-bis(isocyanatomethyl)cyclohexane, preferably of isophorone diisocyanate, or containing 5% to 30% by weight of a first residue A from the production of 1,5-pentane diisocyanate and 70% to 95% by weight of a second residue B from the production of xylylene diisocyanate, preferably of m-xylylene diisocyanate.

Stabilization preferably means an increase in the onset temperature relative to residue B alone. Depending on the type of residue B the efficacious amount of residue A may vary. Said amount may be determined for example in preliminary tests by producing mixtures of residue A with the residue B in question in different mixing ratios and then determining their thermal stability by differential scanning calorimetry (DSC) according to DIN EN ISO 11357-1:2017-02. The efficacious amount of residue A may then be directly derived from the mixing ratios for which an increase in the onset temperature for the exothermic reaction of the residue is observed. If two or more exothermic reactions with different onset temperatures occur for a residue B, the one that is decisive is the one having the lowest onset temperature.

### EXAMPLES

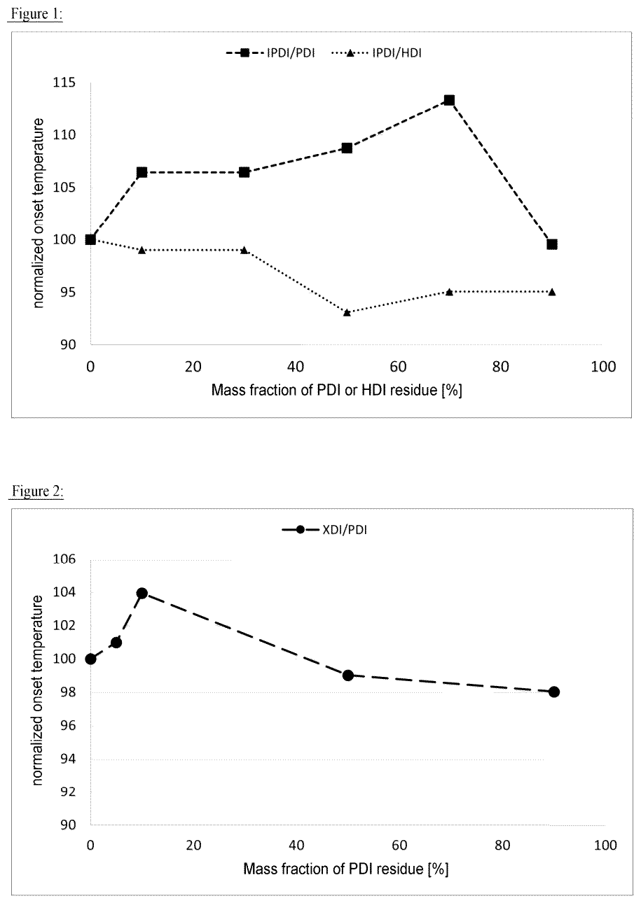

The present invention will now be elucidated with reference to FIG. 1 and FIG. 2 and the examples that follow but is not limited thereto.

For differential scanning calorimetry (DSC) in each case a few milligrams of the residue mixture were weighed into a gold-plated steel crucible under a nitrogen atmosphere and sealed with a crucible press. The samples prepared in this way were then analyzed in the DSC analytical apparatus against a reference crucible filled with inert aluminum oxide. The analytical apparatus met the requirements of DIN EN ISO 11357-1:2017-02. Both crucibles were heated at a heating rate of 3K/min (so-called screening DSC) and the difference between the heat flux into the crucible with the residue mixture and the heat flux into the reference crucible was recorded. According to DIN EN ISO 11357-1:2017-02, in an exothermic peak, i.e. a reaction with release of heat, the heat flux into the test specimen crucible is lower than that into the reference crucible and the recorded curve accordingly passes through a minimum. To assess thermal stability the onset temperature of the first exothermic peak whose release of heat exceeded a value of 100 J/g, i.e. the temperature at which the recorded heat flux difference began to diverge from the baseline of the test specimen, was determined.

### Example 1

A residue from a distillation of isophorone diisocyanate (IPDI) was mixed in different mixing ratios with a residue from a distillation of pentamethylene diisocyanate (PDI). The mixing ratios of IPDI mass fraction: PDI mass fraction in the residue mixtures were 100:0, 90:10, 70:30, 50:50, 30:70 and 10:90. The mixtures were then analyzed by differential scanning calorimetry and the onset temperatures for the exothermic reaction of the respective residue mixture were determined.

### Example 2

A residue from a distillation of isophorone diisocyanate (IPDI) was mixed in different mixing ratios with a residue from a distillation of hexamethylene diisocyanate (HDI). The mixing ratios of IPDI mass fraction: HDI mass fraction in the residue mixtures were 100:0, 90:10, 70:30, 50:50, 30:70 and 10:90. The mixtures were then analyzed by differential scanning calorimetry and the onset temperatures for the exothermic reaction of the respective residue mixture were determined.

### Discussion of Examples 1 and 2

The onset temperatures from Example 1 and Example 2 were respectively normalized so that the 100:0 mixture, i.e. a distillation residue from an IPDI distillation, attained the value 100. The thus-normalized onset temperatures are recorded in FIG. 1.

In the diagram of FIG. 1 the normalized onset temperature was plotted on the Y-axis and the mass fraction of PDI or HDI residue was plotted on the X-axis. The graph with the square data points represents the result of example 1 (IPDI/PDI) and the graph with the triangular data points represents the result of example 2 (IPDI/HDI).

From the graphs in FIG. 1 it is apparent that the mixture with PDI in the range from about 10% by weight PDI content to about 80% by weight PDI content leads to an increase in the onset temperature and thus to a stabilization of the residue mixture while none of the test mixtures exhibits a normalized onset temperature>100 due to admixture of HDI. On the contrary, the normalized onset temperature passes through a minimum and so admixing HDI residue leads to a destabilization of the IPDI residue mixture.

### Example 3

A residue from a distillation of m-xylylene diisocyanate (XDI) was mixed in different mixing ratios with a residue from a distillation of pentamethylene diisocyanate. The mixing ratios of XDI content: PDI content in the residue mixtures were 100:0, 95:5 90:10, 50:50 and 10:90. The mixtures were then investigated by differential scanning calorimetry. The normalized onset temperatures (onset temperature of pure XDI residue normalized to a value of 100) are shown in FIG. 2.

In the diagram of FIG. 2 the normalized onset temperature was plotted on the Y-axis and the mass fraction of PDI residue was plotted on the X-axis. The graph with the round data points represents the result of example 3 (XDI/PDI).

From the graphs in FIG. 2 it is apparent that the mixture with PDI residue in the range from about 5% by weight PDI content to about 30% by weight PDI content leads to an increase in the normalized onset temperature and thus to a stabilization of the residue mixture.

1. A process for treatment of residues from the production of organic isocyanates by phosgenation of organic amines, comprising a mixing step of a first residue A with a second residue B to obtain a residue mixture, wherein residue A is obtained from a production of 1,5-pentane diisocyanate and residue B is obtained from a production of an isocyanate distinct from 1,5-pentane diisocyanate.

2. The process of claim 1, wherein residue A or residue B is a distillation residue.

3. The process of claim 1, wherein residue B derives from the production of at least one araliphatic or cycloaliphatic diisocyanate, distinct from 1,5-pentane diisocyanate.

4. The process of claim 1, wherein residue B derives from the production of hexamethylene diisocyanate, diisocyanatohexylmethane, isophorone diisocyanate, bis(isocyanatomethyl)norbornane, 1,3- and 1,4-bis(isocyanatomethyl)cyclohexane or xylylene diisocyanate.

5. The process of claim 1, wherein the temperature in the mixing step is between 15° C. and 180° C.

6. The process of claim 1, wherein the mixing step affords at least one residue mixture which is stored in at least one residue container, wherein the mixing step is carried out in a separate mixing apparatus upstream of the residue container or in the residue container itself.

7. The process of claim 6, wherein the temperature during storage is between 15° C. and 180° C.

8. The process of claim 6, wherein the at least one residue mixture is continuously stirred, in at least one residue container.

9. The process of claim 1, wherein the mixing step of an efficacious amount of residue A with residue B results in a stabilization of residue B, wherein the stabilization entails an increase in the onset temperature relative to residue B alone.

10. The process of claim 1, wherein 10 to 80 parts by weight of residue A are mixed with 20 to 90 parts by weight of residue B, wherein residue B is from the production of isophorone diisocyanate, bis(isocyanatomethyl)norbornane (NBDI) or 1,3- and 1,4-bis(isocyanatomethyl)cyclohexane.

11. The process of claim 1, wherein the residue supplied to an incineration.

12-14. (canceled)

15. A stabilized residue mixture comprising 10% to 80% by weight of a first residue A from the production of 1,5-pentane diisocyanate and 20% to 90% by weight of a residue B from the production of isophorone diisocyanate, bis(isocyanatomethyl)norbornane (NBDI) or 1,3- and 1,4-bis(isocyanatomethyl)cyclohexane.

16. A stabilized residue mixture comprising 5% to 30% by weight of a first residue A from the production of 1,5-pentane diisocyanate and 70% to 95% by weight of a second residue B from the production of xylylene diisocyanate.

17. The process of claim 1, wherein residue A and residue B are distillation residues.

18. The process of claim 1, wherein residue B derives from the production of isophorone diisocyanate or m-xylylene diisocyanate.

19. The process of claim 1, wherein the temperature in the mixing step is between 40° C. and 150° C.

20. The process of claim 1, wherein the temperature in the mixing step is between 60° C. and 110° C.

21. The process of claim 6, wherein the temperature during storage is between 40° C. and 150° C.

22. The process of claim 6, wherein the temperature during storage is between 60° C. and 110° C.

23. The process of claim 1, wherein 5 to 30 parts by weight of residue A are mixed with 70 to 95 parts by weight of residue B, wherein residue B is from the production of xylylene diisocyanate.
